(19) United States
(12) Patent Application Publication
(10) Pub. No.: US 2026/0335064 A1
(43) Pub. Date: Oct. 29, 2026

(54) DOCKING STATIONS FOR PROSTHETIC IMPLANTS

(71) Applicant: Edwards Lifesciences Corporation, Irvine, CA (US)

(72) Inventors: Richard Leechung Li, Irvine, CA (US); Walter Lee, Irvine, CA (US); Alison Louise Rodriguez, Irvine, CA (US); Nguyen Thuong Nguyen, Orange, CA (US); Christopher William Casalaspi, Irvine, CA (US); Mai Loan Truong, Westminster, CA (US); Thu-Thao Thi Le, Garden Grove, CA (US)

(21) Appl. No.: 19/696,788

(22) Filed: Jun. 3, 2026

Related U.S. Application Data
(63) Continuation of application No. PCT/US2024/056555, filed on Nov. 19, 2024.
(60) Provisional application No. 63/607,036, filed on Dec. 6, 2023.

(51) Int. Cl. A61F 2/02

(52) U.S. Cl. CPC A61F 2/02; A61F 2220/0025

(57) ABSTRACT

A docking station for a prosthetic implant includes a frame. The frame includes one or more tissue engaging elements, which in some examples are apices of the frame formed by struts of the frame. The frame and/or the apices may be configured to engage native tissue at an implantation location to retain the position of the frame without damaging the native tissue. A docking station may also include a sealing skirt coupled to the frame. The sealing skirt may reduce retrograde blood flow through and/or around the frame. The docking station may include a chord of material spanning between at least two apices to prevent undesirable or excessive penetration of the native tissue. The docking station may include strut-wrapping material that encloses two terminal struts leading to an apex of the docking station while leaving the apex exposed.

### CROSS-REFERENCE TO RELATED APPLICATIONS

This application is a continuation of PCT Patent Application No. PCT/US2024/05655, filed on Nov. 19, 2024, which claims priority to and the benefit of United States Utility Patent Application No. 63/607,036, filed Dec. 6, 2023, entitled “DOCKING STATIONS FOR PROSTHETIC IMPLANTS”, each of these applications being incorporated herein in its entirety by this reference.

### FIELD

The present disclosure relates generally to implantable prosthetic devices and more particularly to docking stations for prosthetic heart valves.

### BACKGROUND

The human heart may suffer from various valvular diseases. These valvular diseases may result in significant malfunctioning of the heart and ultimately require repair of the native valve or replacement of the native valve with an artificial valve. There are a number of known repair devices (e.g., stents) and artificial valves, as well as a number of known methods of implanting these devices and valves in humans. Percutaneous and minimally invasive surgical approaches are used in various procedures to deliver prosthetic medical devices to locations inside the body that are not readily accessible by surgery or where access without surgery is desirable.

In one specific example, a prosthetic valve may be mounted in a crimped state on the distal end of a delivery apparatus and advanced through the patient’s vasculature (e.g., through a femoral artery and the aorta) until the prosthetic valve reaches the implantation location in the heart. The prosthetic valve is then expanded to its functional size, for example, by inflating a balloon on which the prosthetic valve is mounted, actuating a mechanical actuator that applies an expansion force to the prosthetic valve, or by deploying the prosthetic valve from a sheath of the delivery apparatus so that the prosthetic valve may self-expand to its functional size.

In some cases, it may not be possible to secure the prosthetic valve to the native valve annulus, for example, if the native valve annulus is too large or if the geometry of the native valve is too complex to allow secure implantation of the valve. One approach in these cases is to first deploy a docking station at the implantation location and then install the prosthetic valve in the docking station. The docking station may be selected to provide the necessary interface to anchor the prosthetic valve within the native valve annulus. Desirably, the docking station may be delivered to the implantation location with a minimally invasive procedure, which would allow the docking station to be deployed within the same procedure used to deliver the prosthetic valve.

### SUMMARY

Disclosed herein are examples of prosthetic implants, such as docking stations, which may be implanted within a patient’s body. The disclosed docking stations can, for example, be positioned within or adjacent a native heart valve annulus and be configured to receive a prosthetic heart valve. In this manner, the docking stations act as a support structure or anchor to help retain the positioning of the prosthetic heart valve relative to the native anatomy. The disclosed docking stations may comprise a frame comprising a plurality of struts, and the plurality of struts may form a set of apices. The docking station may also include a chord of material spanning between at least two apices of the set of apices.

Another example embodiment included in the present disclosure includes a method that includes obtaining a frame, where the frame includes multiple struts, and where the struts form a set of apices. The method may also include threading a chord between eyelets of at least two apices of the set of apices; and coupling the chord to the frame.

Another example embodiment included in the present disclosure includes a docking station for a prosthetic implant that includes a frame with multiple struts, where the struts form apices. The docking station may also include strut-wrapping material enclosing two terminal struts of the multiple struts, where the two terminal struts lead up to an apex of the apices formed by the two terminal struts. The strut-wrapping material may leave the apex exposed.

The various innovations of this disclosure may be used in combination or separately. This summary is provided to introduce a selection of concepts in a simplified form that are further described below in the detailed description. This summary is not intended to identify key features or essential features of the claimed subject matter, nor is it intended to be used to limit the scope of the claimed subject matter. The foregoing and other objects, features, and advantages of the disclosure will become more apparent from the following detailed description, claims, and accompanying figures.

### BRIEF DESCRIPTION OF THE DRAWINGS

FIG. 1 is an elevation view of a portion of a frame of a docking station in a radially-expanded state.

FIG. 2 is a perspective view of the frame of FIG. 1 in a radially-compressed state.

FIG. 3 is a perspective view of a docking station including the frame of FIG. 1.

FIG. 4 is a cut-away view of the docking station of FIG. 3 deployed at an implantation location within a patient’s anatomy, which is depicted schematically in cross-section, and with a prosthetic heart valve deployed therein.

FIG. 5A is a perspective view of a delivery apparatus for deploying a docking station.

FIG. 5B illustrates the docking station of FIG. 3 disposed around a distal portion of the delivery apparatus of FIG. 5A.

FIG. 6A is an elevation view of a distal portion of the delivery apparatus of FIG. 5A with an outer shaft of the delivery apparatus in a retracted position.

FIG. 6B is an elevation view of a distal portion of the delivery apparatus of FIG. 5A with an outer shaft of the delivery apparatus in an extended position and cut away to show an encapsulated docking station.

FIGS. 6C-6F illustrate stages in deployment of the docking station of FIG. 3 from the delivery apparatus of FIG. 5A.

FIG. 7A is a perspective view of a handle portion of the delivery apparatus illustrated in FIG. 5A.

FIGS. 7B and 7C are perspective views of the handle portion of FIG. 7A with a portion of the handle cut away to show various internal components.

FIG. 8 is a perspective view of an example docking station with chords between apices.

FIGS. 9A-9D illustrate various examples of an apex associated with a chord.

FIG. 10 is a perspective view of another example docking station with chords between apices.

FIGS. 11A-11D illustrate various example configurations of chords and apices of a docking station.

FIG. 12A is a perspective view of another example docking station with strut-wrapping material wrapped around terminal struts of the docking station.

FIG. 12B is a focused view on a single apex of the docking station of FIG. 12A.

### DETAILED DESCRIPTION

### General Considerations

For purposes of this description, certain aspects, advantages, and novel features of examples of this disclosure are described herein. The disclosed methods, apparatus, and systems should not be construed as being limiting in any way. Instead, the present disclosure is directed toward all novel and nonobvious features and aspects of the various disclosed examples, alone and in various combinations and sub-combinations with one another. The methods, apparatus, and systems are not limited to any specific aspect or feature or combination thereof, nor do the disclosed examples require that any one or more specific advantages be present or problems be solved.

Although the operations of some of the disclosed examples are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods may be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

In the interest of conciseness, and for the sake of continuity in the description, same or similar reference characters may be used for same or similar elements in different figures, and description of an element in one figure will be deemed to carry over when the element appears in other figures with the same or similar reference character. In some cases, the term “corresponding to” may be used to describe correspondence between elements of different figures. In an example usage, when an element in a first figure is described as corresponding to another element in a second figure, the element in the first figure is deemed to have the characteristics of the other element in the second figure, and vice versa, unless stated otherwise.

As used in this application and in the claims, the singular forms “a,” “an,” and “the” include the plural forms unless the context clearly dictates otherwise. The word “comprise” and derivatives thereof, such as “comprises” and “comprising,” are to be construed in an open, inclusive sense, that is, as “including, but not limited to.” Additionally, the term “includes” means “comprises.” Further, the term “coupled” generally means physically, mechanically, chemically, magnetically, and/or electrically coupled or linked and does not exclude the presence of intermediate elements between the coupled or associated items absent specific contrary language.

As used herein, the term “proximal” refers to a position, direction, or portion of a device that is closer to the user and further away from the implantation site. As used herein, the term “distal” refers to a position, direction, or portion of a device that is further away from the user and closer to the implantation site. Thus, for example, proximal motion of a device is motion of the device away from the implantation site and toward the user (e.g., out of the patient’s body), while distal motion of the device is motion of the device away from the user and toward the implantation site (e.g., into the patient’s body). The terms “longitudinal” and “axial” refer to an axis extending in the proximal and distal directions, unless otherwise expressly defined.

As used herein, the term “simulation” means a performing an act on a cadaver, cadaver heart, anthropomorphic ghost, and/or a computer simulator (e.g., with the body parts, tissue, etc. being simulated).

### Introduction to the Disclosed Technology

As mentioned above, the docking stations disclosed herein may comprise a frame having a plurality of struts. The struts of the frame can, in some instances, form one or more apices at the inflow and/or outflow ends of the frame. In some implementations, the frame may include one or more features configured to help retain the docking station relative to the native anatomy. For example, the frame may comprise a contoured shape (e.g., an hourglass shape) corresponding to the shape of the native anatomy. Additionally or alternatively, the apices of the frame may engage the native tissue to help prevent or reduce migration of the docking station relative to the native tissue.

Although engagement between the frame (e.g., the apices) and the native tissue may be beneficial for preventing migration of the docking station relative to the native tissue, it may also be beneficial to control the extent to which the frame engages the native tissue. For example, limiting or reducing engagement between the frame and the native tissue can, for instance, help prevent the frame from puncturing, tearing, and/or otherwise damaging the native tissue.

Thus, disclosed herein are various frame configurations (e.g., shapes) and/or other features configured to allow the docking station to have sufficient anti-migration relative to the native anatomy, while also reducing the likelihood of tissue damage. For example, the docking stations disclosed herein may comprise one or more covers and/or frame shapes configured to prevent or reduce the likelihood that the frame (e.g., the apices) will damage the native tissue.

### Examples of the Disclosed Technology

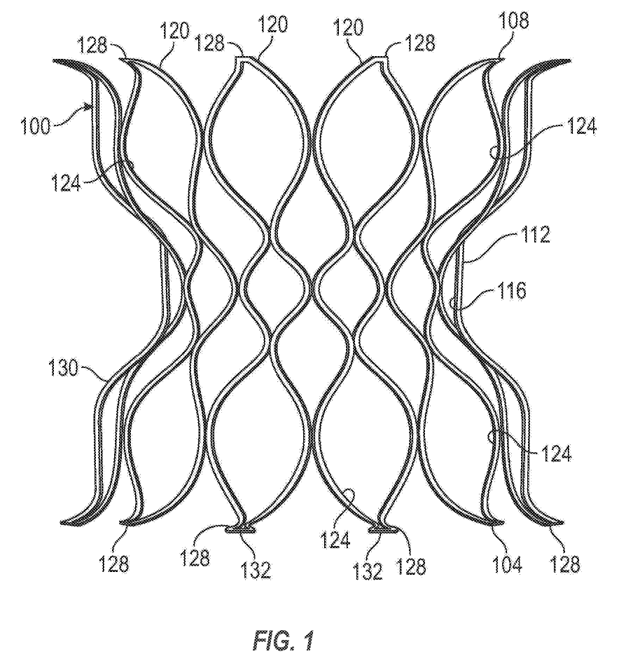

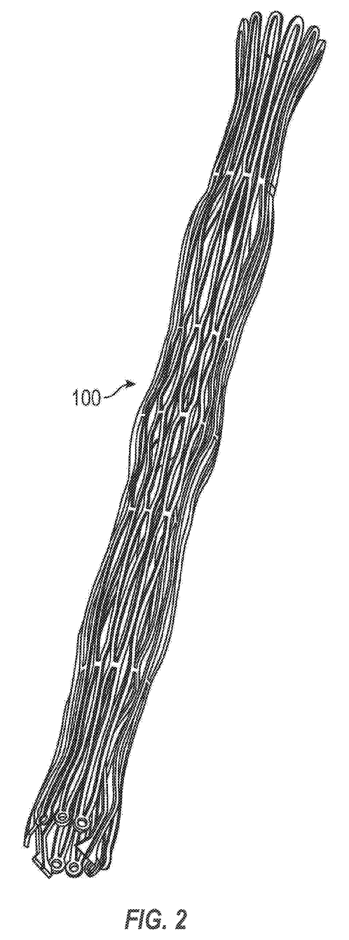

Turning now to the drawings, FIG. 1 illustrates an example implementation of a frame 100 (or stent) that may form a body of a docking station. The frame 100 has a first end 104 and a second end 108. In some examples, the first end 104 may be an inflow end, and the second end 108 may be an outflow end. In some examples, the first end 104 may be an outflow end, and the second end 108 may be an inflow end. The terms “inflow” and “outflow” are related to the normal direction of blood flow (e.g., antegrade blood flow) through the frame. In the unconstrained, expanded state of the frame 100 shown in FIG. 1, a relatively narrower portion (or waist) 112 of the frame 100 between the first end 104 and the second end 108 forms a valve seat 116. The frame 100 may be compressed (as illustrated in FIG. 2) for delivery to an implantation location by a delivery apparatus.

Although the docking stations, delivery apparatus, prosthetic heart valves, and/or methods are described herein with respect to a particular implantation location (e.g., a pulmonary valve) and/or a particular delivery approach (e.g., transfemoral), the device and methods disclosed herein may be adapted to various other implantation locations (e.g., an aortic valve, a mitral valve, or a tricuspid valve) and/or delivery approaches (e.g., transapical, transseptal, etc.).

In the example illustrated by FIG. 1, the frame 100 includes a plurality of struts 120 arranged to form cells 124. The ends of the struts 120 form apices 128 at the ends of the frame 100. One or more of the apices 128 may include a connector tab 132. The portions of the struts 120 between the apices 128 and the valve seat 116 (or the waist 112) form a sealing portion 130 of the frame 100. In the unconstrained, expanded state of the frame 100 illustrated in FIG. 1, the apices 128 extend generally radially outward and are radially outward of the valve seat 116.

The frame 100 may be made of a highly resilient or compliant material to accommodate large variations in the anatomy. For example, the frame 100 may be made of a flexible metal, metal alloy, polymer, or an open cell foam. An example of a highly resilient metal is nitinol, which is a metal alloy of nickel and titanium, but other metals and high resilient or compliant non-metal materials may be used. The frame 100 may be self-expanding, manually expandable (e.g., expandable via a balloon), or mechanically expandable. A self-expanding frame may be made of a shape memory material, such as, for example, nitinol. In this manner, the frame may be radially compressed as depicted in FIG. 2 (e.g., via a crimping device) and may radially expand to the configuration depicted in FIG. 1.

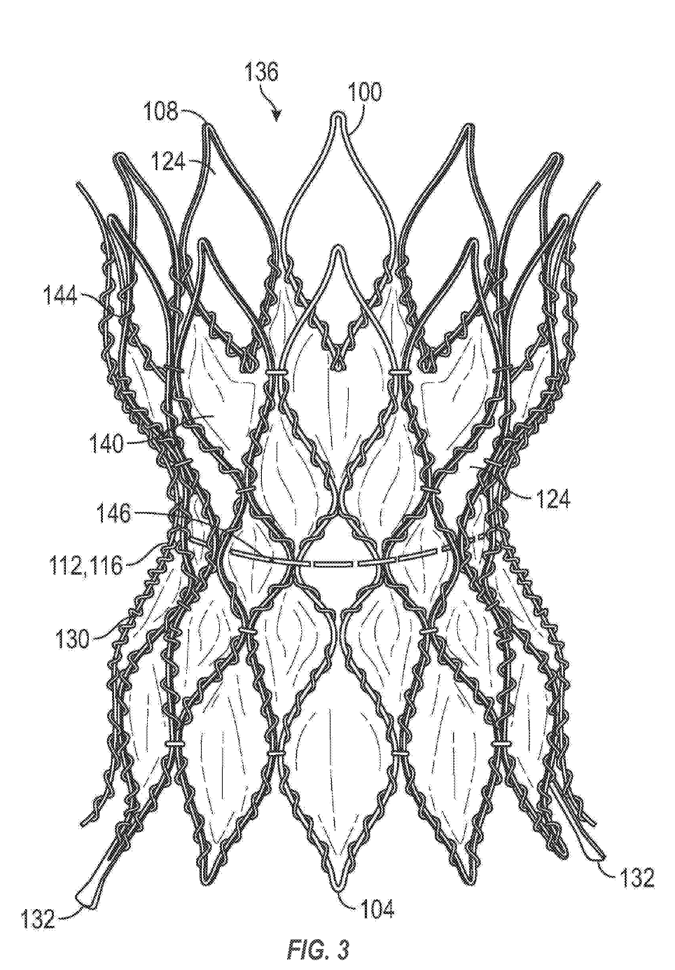

FIG. 3 illustrates an example docking station 136 including the frame 100 and a sealing skirt 140 disposed within the frame. The sealing skirt 140 is attached to the frame 100 (e.g., by sutures 144). In the example illustrated by FIG. 3, the sealing skirt 140 covers at least the cells 124 in the sealing portion 130 of the frame 100. In this manner, the sealing skirt 140 may help funnel blood flowing into the docking station 136 from the proximal inflow end 104 to the valve seat 116 (and the valve once installed in the valve seat). The sealing skirt may additionally or alternatively help to prevent or reduce parastent leakage (e.g., retrograde blood flow between the docking station and the native tissue and/or paravalvular leakage (e.g., retrograde blood flow between the prosthetic heart valve and the docking station. In the illustrated example, the row of cells proximate to the distal outflow end 108 is not covered by the sealing skirt 140. The uncovered cells can, for example, permit blood to flow through the distal side of the frame and/or enable catheter access to the left and right pulmonary arterial branches (e.g., to pass another intravascular device (e.g., catheter, wire, etc.) during the implantation procedure and/or during a secondary intervention).

The sealing skirt 140 may be a fabric that is impermeable to blood. A variety of biocompatible materials may be used for the sealing skirt 140, such as, for example, foam or a fabric that is treated with a coating that is impermeable to blood, a polyester material, or a processed biological material, such as pericardium. In some examples, the sealing skirt 140 may comprise polymeric material, including polyethylene terephthalate (PET), expanded polytetrafluorethylene (ePTFE), and/or thermoplastic polyurethane (TPU).

The docking station 136 may include a band 146 that extends around the waist 112 (or that is integral to the waist) of the frame 100. The band 146 may constrain expansion of the valve seat 116 to a specific diameter in the deployed state to enable the valve seat 116 to support a specific valve size. The band 146 may take on a wide variety of different forms and may be made of a wide variety of different materials. For example, the band 146 may be made of PET, one or more sutures, fabric, metal, polymer, a biocompatible tape, or other relatively nonexpanding materials known in the art and that may maintain the shape of the valve seat 116.

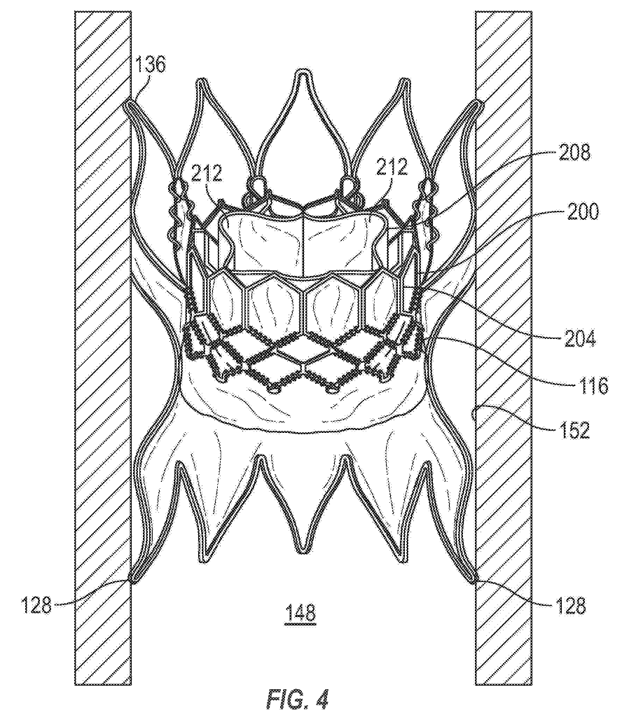

FIG. 4 illustrates the docking station 136 in a deployed state within a native valve annulus 148 (shown schematically). As may be seen, the frame 100 of the docking station 136 is in an expanded condition, with the end portions of the frame pressed against the inner surface 152 of the native valve annulus. The band 146 (shown in FIG. 3) may maintain the valve seat 116 at a constant or substantially constant diameter in the expanded condition of the frame 100. FIG. 4 also shows a prosthetic valve 200 deployed within the docking station 136 and engaged with the valve seat 116 of the docking station 136. The prosthetic valve 200 may be implanted by first deploying the docking station 136 at the implantation location and then installing the prosthetic valve within the docking station.

The prosthetic valve 200 may be configured to replace a native heart valve (e.g., aortic, mitral, pulmonary, and/or tricuspid valves). In one example, the prosthetic valve 200 may include a frame 204 and a valvular structure 208 disposed within and attached to the frame 204. The valvular structure 208 may include one or more leaflets 212 that cycle between open and closed states during the diastolic and systolic phases of the heart. The frame 204 may be made of the frame materials described for the frame 100 of the docking station 136. The leaflets 212 may be made in whole or in part from pericardial tissue (e.g., bovine pericardial tissue), biocompatible synthetic materials, or various other suitable natural or synthetic materials known in the art.

The docking station 136 is not limited to use with the particular example of the prosthetic valve 200 illustrated in FIG. 4. For example, mechanically expandable prosthetic valves may be installed in the docking station 136. Example mechanically expandable prosthetic valves are described in U.S. Patent No. 10,603,165 and 10,806,573 and International Application Nos. PCT/US2019/056865 and PCT/US2020/040318, which are incorporated by reference herein. Additional information about docking stations and prosthetic valves may be found in U.S. Patent No. 10,363,130, which is incorporated by reference herein.

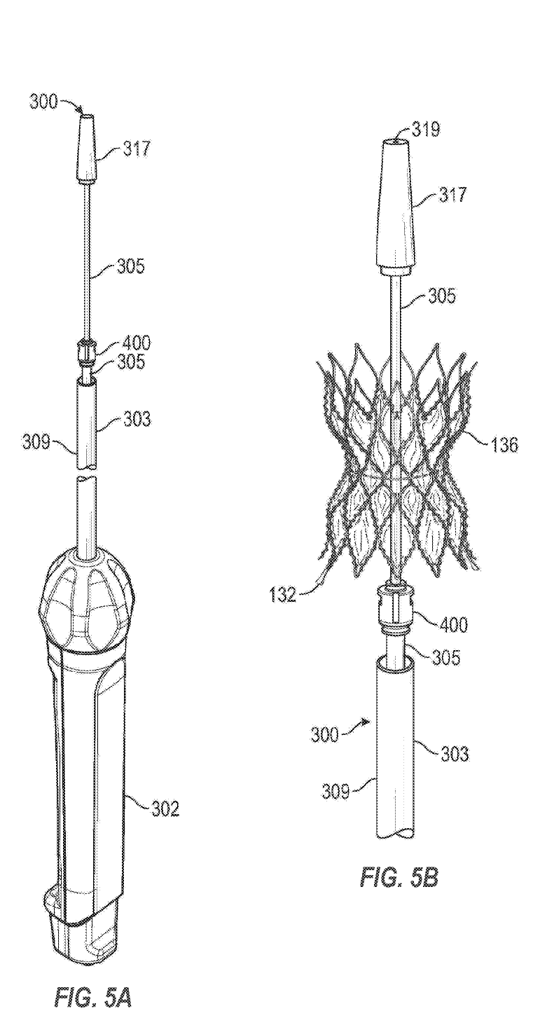

FIG. 5A illustrates an example delivery apparatus 300 that may be used to deliver the docking station to an implantation location. The delivery apparatus 300 generally includes a handle 302 and a shaft assembly 303 coupled to the handle 302 and extending distally from the handle 302. The shaft assembly 303 includes an inner shaft 305 and an outer shaft 309. The inner shaft 305 extends through a lumen of the outer shaft 309.

In the example illustrated by FIG. 5A, a frame connector 400 is coupled to the inner shaft 305. The docking station 136 may be disposed around a portion of the inner shaft 305 extending distally from the frame connector 400, as shown in FIG. 5B. In one example, the frame connector 400 includes one or more recesses that may receive one or more connector tabs 132 at the proximal end of the docking station 136 and thereby axially restrain the docking station 136.

A nosecone 317 may be attached to a distal end of the inner shaft 305. The nosecone 317 includes a central opening 319 for receiving a guidewire. As such, a proximal end of the guidewire may be inserted into the central opening 319 and through the inner shaft 305, and a distal end portion of the delivery apparatus 300 may be advanced over the guidewire through a patient’s vasculature and to an implantation location. The guidewire may pass through the nosecone 317 into the inner shaft 305 during advancing of the delivery apparatus through a patient’s vasculature.

The handle 302 may be operated to move the outer shaft 309 relative to the inner shaft 305, generally between an extended position and a retracted position. The handle 302 may be extended to slide the outer shaft 309 over the frame connector 400 and over any docking station coupled to the frame connector 400 to encapsulate the docking station within the outer shaft 309. As the outer shaft 309 slides over the docking station 136, the outer shaft 309 may compress the docking station 136 such that the docking station is encapsulated within the outer shaft 309 in the compressed state. In the fully extended position, a distal end of the outer shaft 309 may abut a proximal end of the nosecone 317 such that there are no gaps in the delivery assembly. Additionally or alternatively, a crimping device may be used to radially compress the docking station such that it may be inserted into the outer shaft of the delivery apparatus.

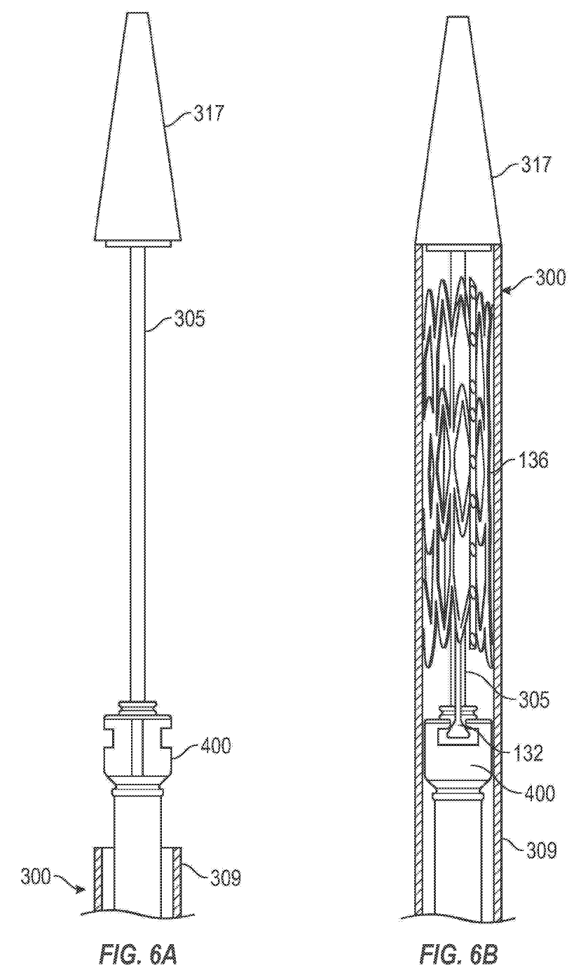

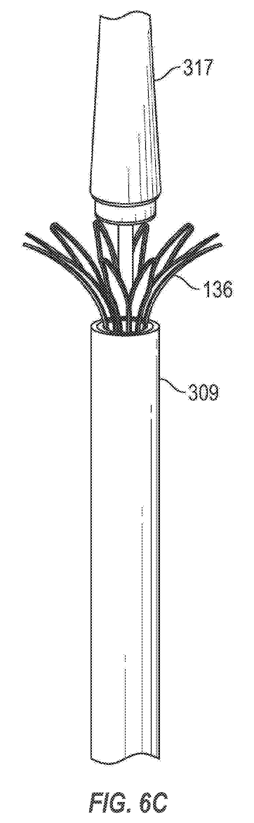

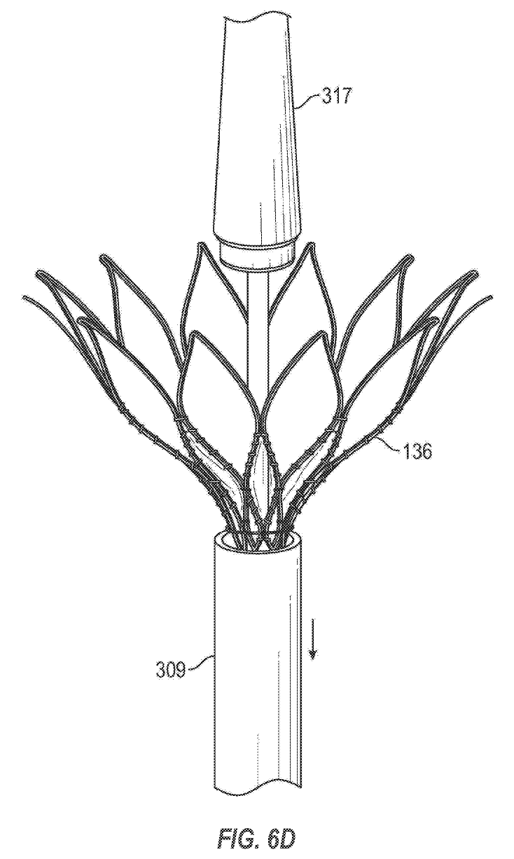

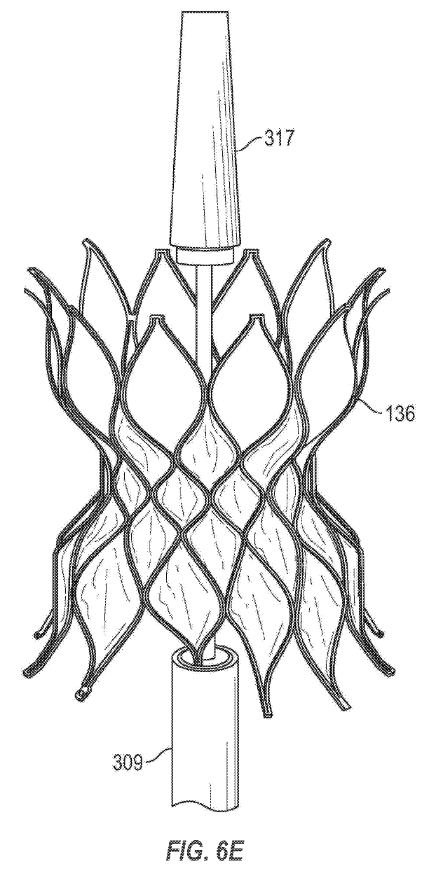

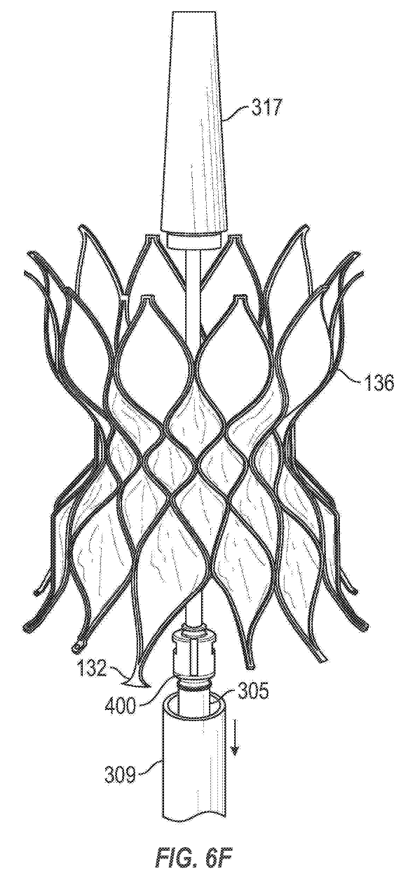

FIGS. 6A-6F illustrate a method of deploying a docking station at an implantation location within an anatomy. For purposes of illustration, the patient’s anatomy is omitted. In FIG. 6A, the method includes retracting the outer shaft 309 by the handle of the delivery apparatus to allow loading of the docking station 136 onto the inner shaft 305. In FIG. 6B, the method includes disposing the docking station 136 around the inner shaft 305 and engaging each of the connector tabs 132 of the docking station 136 with the frame connector 400. The method also includes positioning the outer shaft 309 over the docking station such that the docking station is encapsulated therein. This may be accomplished by manipulating the handle of the delivery apparatus. As shown in FIG. 6B, the distal end of the outer shaft 309 abuts the proximal end of the nosecone 317. The method includes inserting the delivery apparatus, from the nosecone 317 end, into a patient’s vasculature and advancing the delivery apparatus through the patient’s vasculature to the implantation location.

At the implantation location, the method includes retracting the outer shaft 309 by the handle of the delivery apparatus to expose the docking station 136. FIGS. 6C-6F show different stages of retracting the outer shaft 309. As may be seen, in cases where the docking station 136 is self-expanding, the docking station 136 gradually emerges from the outer shaft 309 and gradually expands from the compressed state as the outer shaft 309 is retracted. When the outer shaft 309 is sufficiently retracted, the connector tabs 132 disengage from the frame connector 400. Once the docking station 136 is disengaged from the frame connector 400, the docking station 136 may radially expand to engage the anatomy.

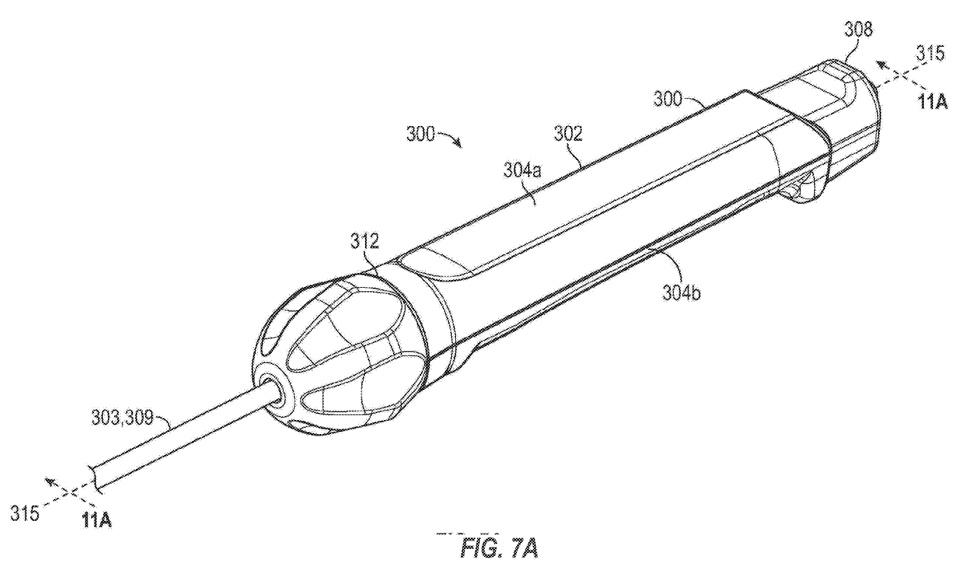

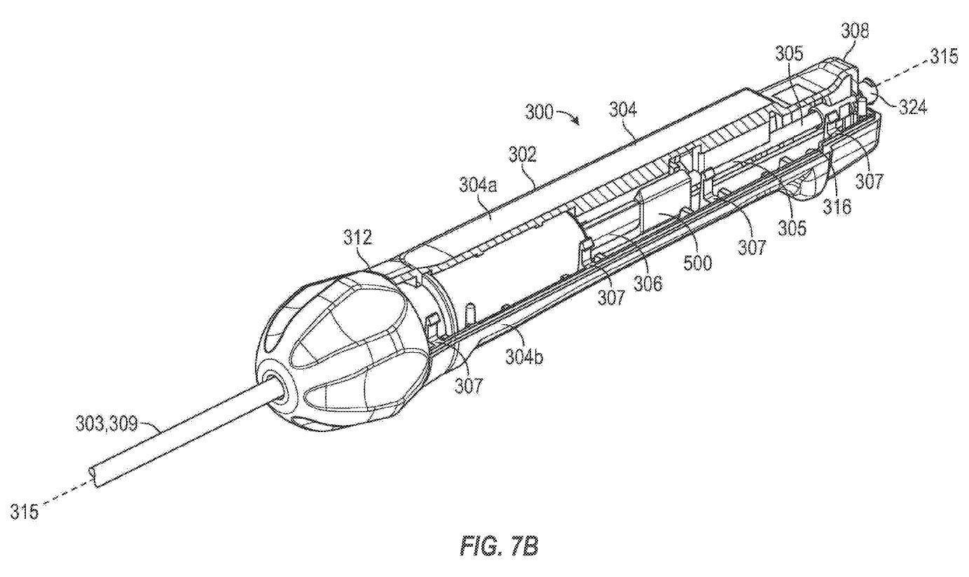

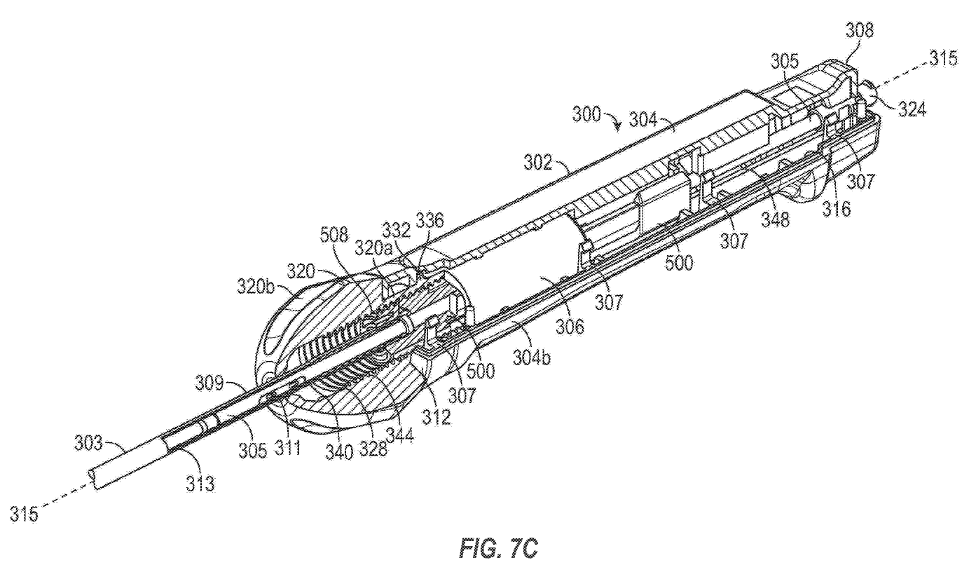

FIGS. 7A-7C illustrate an example implementation of the handle 302 of the delivery apparatus. The handle 302 includes a handle body 304 and a deployment mechanism 306 coupled to and partially disposed within the handle body. The handle body 304 includes a proximal end 308, a distal end 312, and a cavity 316 extending from the proximal end 308 to the distal end 312. The handle 302 includes a longitudinal axis 315 extending from the proximal end 308 to the distal end 312. The longitudinal axis 315 defines the axial direction of the handle.

The handle body 304 may be a single piece body with the cavity 316. Alternatively, the handle body 304 may have two body pieces 304a, 304b that may be assembled together to form the cavity 316. For example, the first body piece 304b may have snap hooks 307 that snap into complementary recesses in the second body piece 304a.

The deployment mechanism 306 of the handle 302 includes a carriage member 500 and a drive member 320. The carriage member 500 is disposed within the cavity 316 and movable relative to the handle body 304 in the axial direction. The drive member 320 engages with the carriage member 500 and is movable (e.g., rotatable) relative to the handle body 304 to adjust the axial position of the carriage member 500 relative to the handle body 304.

Proximal portions of the shafts 305, 309 are inserted into the cavity of the handle body 304. A proximal end portion of the outer shaft 309 of the shaft assembly 303 may be coupled to the carriage member 500 (e.g., by fasteners, adhesive, and/or other means for coupling) such that movement of the carriage member 500 relative to the handle body 304 causes movement of the outer shaft 309 between the extended and retracted positions.

A proximal portion of the inner shaft 305 extends through a lumen 313 of the outer shaft 309 into a proximal portion of the cavity 316 and is coupled to the handle body 304. The inner shaft 305 may be fixed relative to the handle body 304 such that the inner shaft 305 is stationary while the outer shaft 309 moves relative to the handle body 304.

In the example illustrated by FIGS. 7A-7C, an injection port 324 is mounted at an opening at the proximal end 308 of the handle body 304. The injection port 324 may be, for example, a Luer fitting. A proximal end of the inner shaft 305 may be inserted into the injector port 324 and secured to the injection port 324 (e.g., by bonding). In some cases, the attachment of the inner shaft 305 to the injection port 324 may serve the purpose of fixing the inner shaft 305 relative to the handle body 304.

The injection port 324 may be used to inject flushing fluid, such as saline, into the lumen of the inner shaft 305. In some cases, the inner shaft 305 may include one or more fluid ports 311 through which the injected fluid exits the inner shaft 305 and enters the lumen 313 of the outer shaft 309, thereby allowing flushing of the lumens of the inner shaft 305 and outer shaft 309 from a single injection port.

Additional details regarding the delivery apparatus and its components may be found in U.S. Application Nos. 63/154,956 and 63/154,966 and International Application No. PCT/US2022/018093, which are incorporated by reference herein.

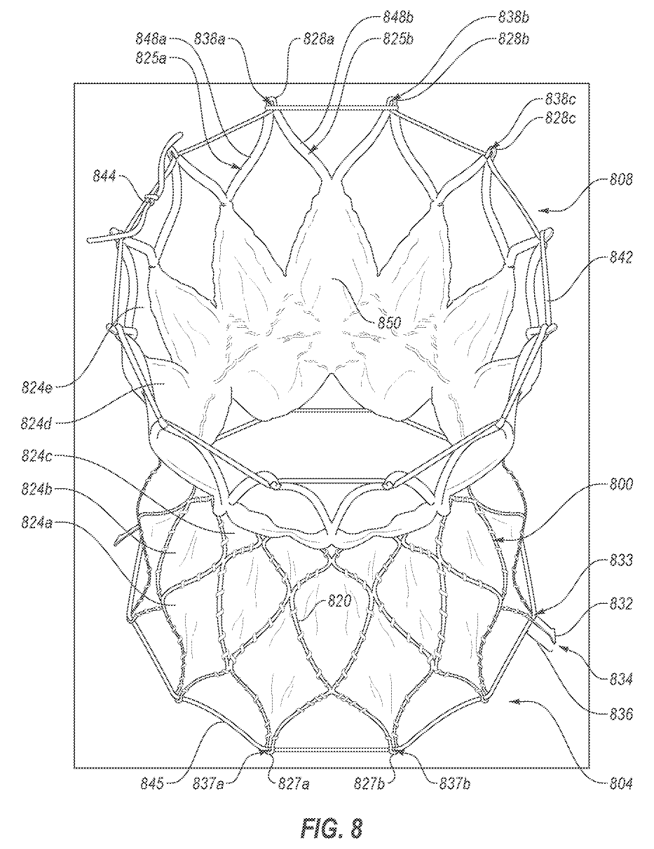

FIG. 8 illustrates an example implementation of a frame 800 (or stent) that may form a body of a docking station that may be similar or comparable to the frame 100. The frame 800 may include a first end 804 and a second end 808, which may be similar or comparable to the first end 104 and the second end 108. In some examples, the first end 804 may be an inflow end, and the second end 808 may be an outflow end. In some examples, the first end 804 may be an outflow end, and the second end 808 may be an inflow end. The terms “inflow” and “outflow” are related to the normal direction of blood flow (e.g., antegrade blood flow) through the frame. In the unconstrained, expanded state of the frame 800 shown in FIG. 8, the frame 800 forms a valve seat within which a valve may be deployed into the frame 800.

The frame 800 may include struts 820 (which may be similar or comparable to the struts 120) which form cells 824 (which may be similar or comparable to the cells 124), such as the rows of cells 824a-824e. At the first end 804 of the frame 800, the struts 820 may form apices 827, such as the apices 827a and 827b. At the second end 808 of the frame 800, the struts 820 may form apices 828, such as the apices 828a, 828b, and 828c. The apices 827 and 828 maybe similar or comparable to the apices 128. With reference to the second end 808, terminal struts 825 may come together to form the apices 828 (such as the terminal struts 825a and 825b coming together to form the apex 828a). In some embodiments the apices 827 and 828 may be characterized as inflow apices or outflow apices, depending on the orientation of the frame 800 relative to the direction of blood flow.

The frame 800 may include a sealing skirt 850 disposed within the frame 800. The sealing skirt 850 maybe similar or comparable to the sealing skirt 140. In some embodiments, the sealing skirt 850 may span some or all of the cells 824. For example, the sealing skirt 850 may fill the rows of cells 824a, 824b, 824c, and 824d but may not extend into the row of cells 824e (e.g., the distal-most row of cells). In such an embodiment, the terminal struts 825 may not have any material of the sealing skirt 850 and/or thread for fixing the sealing skirt 850 to the frame 800 associated therewith.

As illustrated in FIG. 8, one or more of the apices may include a chord spanning between the adjacent apices. For example, at the first end 804, a chord 845 of material may span between the apices 827, such as the apices 827a and 827b. As another example, at the second end 808, a chord 842 of material may span between the apices 828, such as the apices 828a, 828b, and 828c. While various details and variations may be described with reference to the chord 842 and the apices 828, it will be appreciated that the same details and variations are equally applicable to the chord 845 and the apices 827.

In some embodiments, the chord 842 may be threaded through eyelets 838 (such as the eyelets 838a, 838b, and 838c) of respective apices 828. For example, after being threaded through the eyelet 838a of the apex 828a, the chord 842 may proceed to the apex 828b where it is threaded through the eyelet 838b, after which the chord 842 may proceed to the apex 828c where it is threaded through the eyelet 838c. When doing so, the chord 842 may be simply threaded through the eyelet 838 before proceeding to a next adjacent or successive apex 828. Additionally or alternatively, the chord 842 may be wound one or more times about the apex 828 before proceeding to the next apex. For example, after threading the chord 842 through the eyelet 838b, the chord 842 may proceed back towards the apex 828a and then wrapped back around the apex 828b before then proceeding to the apex 828c to be threaded through the eyelet 838c.

In some embodiments, after being threaded through all of the applicable apices 828, the chord 842 maybe fixed to the frame 800. For example, the chord 842 may be tied to the frame 800. Additionally or alternatively, when making a loop around the frame 800, one end of the chord 842 may be tied in a knot 844 with the opposite end of the chord 842. Any knot may be used to fix the chord 842 to the frame 800. In some embodiments, instead of or in addition to tying the knot 844, the material of the chord 842 may be sealed to itself, such as by heating or chemical reaction, to make a closed loop of the chord 842.

At the first end 804, the frame 800 may include a connector tab 832 that may be similar or comparable to the connector tab 132. As illustrated in FIG. 8, the connector tab 832 may include an eyelet 833 that is part way along the connector tab 832 rather than at the apex 834 of the connector tab 832. For example, the eyelet 833 may be a distance 836 away from the apex 834 of the connector tab 832. While illustrated as being formed partway along the connector tab 832 rather than at the end of the connector tab 832, it will be appreciated that the eyelet 833 may be formed at the end of the connector tab 832.

In some embodiments, the eyelet 833 may be as close to the first end 804 as the eyelets 837. In such an embodiment, the generally circularity of the chord 845 and the interconnected apices 827 may be maintained while recognizing that the connector tab 832 may puncture the tissue more deeply than the apices 827. Or stated another way, the distance 836 may extend at the first end 804 of the frame 800 beyond the other apices 827. In these and other embodiments, the row of cells 824a may be consistently sized around the entire circumference of the frame 800, including at the connector tab 832.

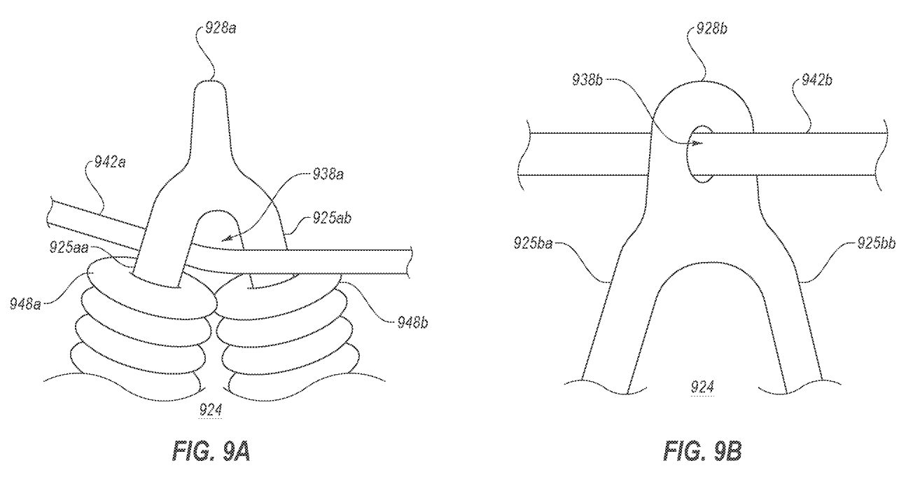

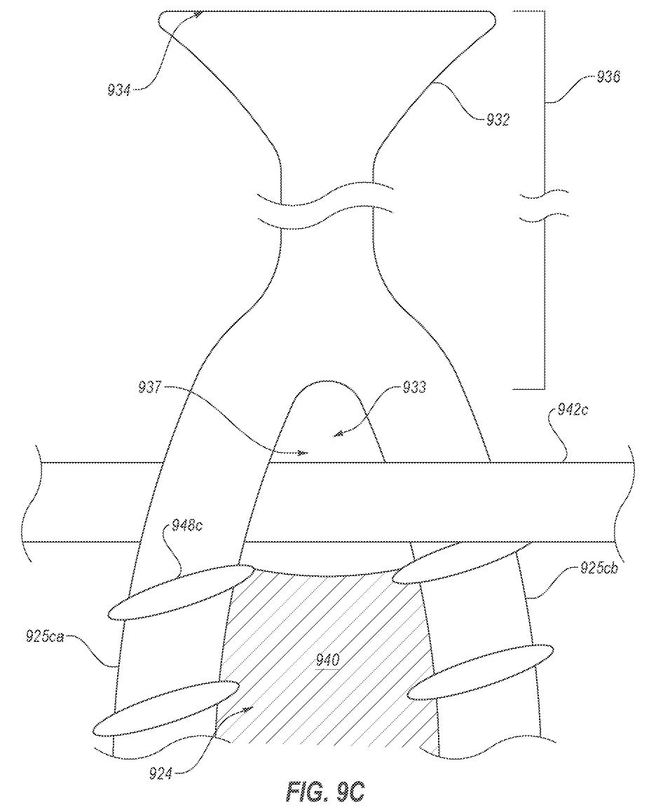

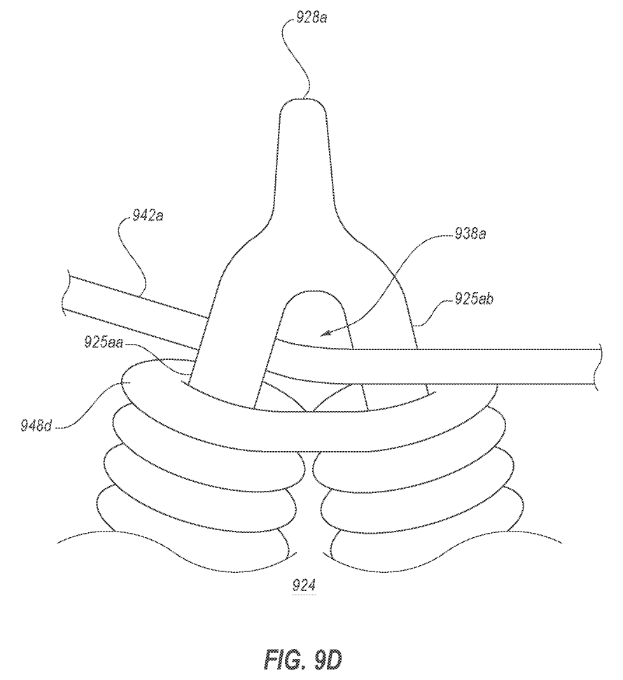

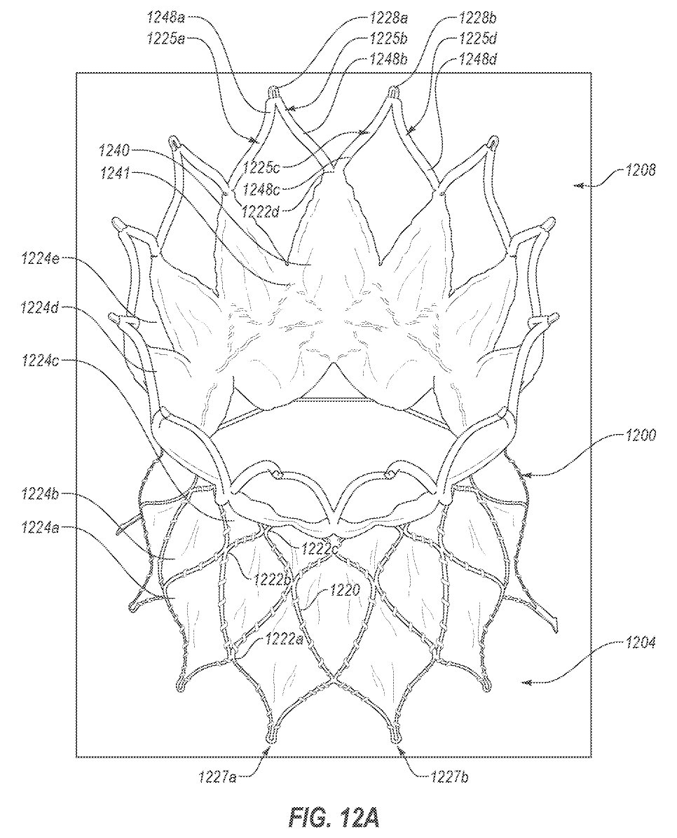

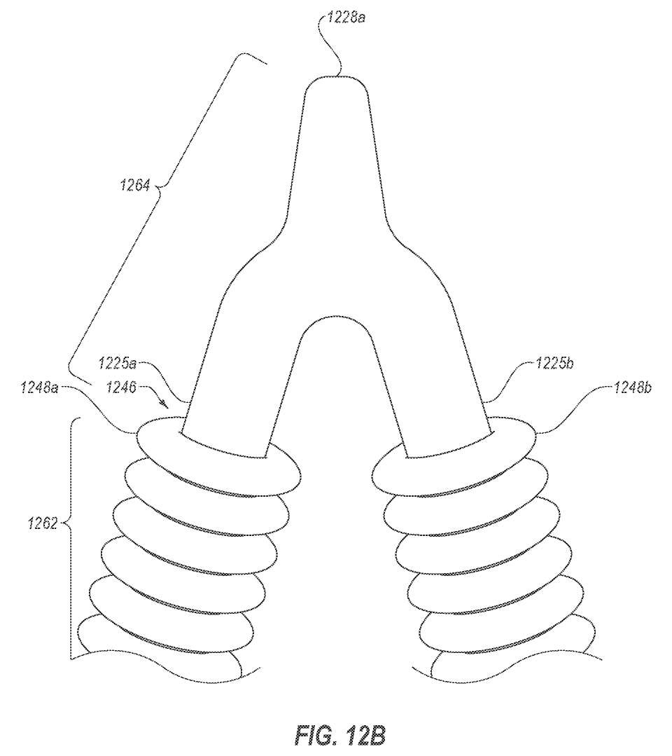

In some embodiments, the eyelets 833, 837, and/or 838 may be formed into the material of the frame 800. Additionally or alternatively, the eyelets 833, 837, and/or 838 may be formed by strut-wrapping material 848 wrapped around the frame 800 and/or the sealing skirt 850. Examples of the eyelets 833, 837, and/or 838 being formed via the strut-wrapping material 848 is illustrated in FIGS. 9A and 9D and an example of the eyelets 833, 837, and/or 838 being formed into the material of the frame 800 is illustrated in FIG. 9B. An example of the strut-wrapping material 848 is described in greater detail with reference to FIGS. 12A-12B. Although FIG. 8 depicts chords 842, 845 and strut-wrapping material 848 in the same embodiment, embodiments herein may alternatively or additionally include one or more chords 842, 845 (or other chords herein) without strut-wrapping material 848 (or other strut-wrapping material) or strut-wrapping material 848 (or other strut-wrapping material) without one or more chords 842, 845 (or other chords herein). As illustrated in FIG. 8, the sealing skirt 850 may fill the row of cells 824a. As the sealing skirt 850 reaches the eyelet 833 of the connector tab 832, the sealing skirt 850 may leave a small gap that forms the eyelet 833. An example of the eyelet 833 associated with the connector tab 832 relative to the sealing skirt 850 is illustrated in FIG. 9C.

By providing the chord 842, the frame 800 may control an amount of tissue penetration when deployed at an implantation location. For example, the chord 842 may increase the surface area between apices 828 such that the frame 800 may resist tissue penetration beyond a depth at which the chord 842 is located along the apices 828. By controlling and/or reducing the amount of tissue penetration by the apices 828, a balance may be found where sufficient tissue penetration occurs to stabilize the frame 800 at the implantation location without causing unacceptable amounts of harm due to erosion of the tissue and/or excessive penetration.

In some embodiments, the chord 842 may facilitate expansion of the frame 800 in a more controlled manner. For example, the chord 842 may connect each of the apices 828 such that they expand in a generally uniform manner (e.g., one apex will not be able to expand too quickly without also pulling along the other apices); stated another way, the chord 842 may help to avoid asymmetric expansion of the apices 828. Because the chord 842 constrains an outer diameter of the apices 828 of the frame 800, the outer diameter may maintain a generally circular shape during expansion of the frame 800. Maintaining the generally circular shape may reduce excessive penetration of tissue by the apices 828 as asymmetrical expansion may result in one (or more) apices that extend further than the others. In such a circumstance, as the other apices penetrate the tissue to stabilize the frame 800 in place, the one or more apices that extended further may penetrate the tissue more deeply than is desirable.

In some embodiments, the chord 842 may reduce an amount of deflection that may occur at the apices 828 when penetrating the tissue during implantation of the frame 800. For example, a given apex may undergo deflection when first penetrating the tissue which may result in fracturing of the frame 800 if too much deflection is experienced. By providing the chord 842, the amount of deflection may be limited as the chord 842 limits how far a given apex 828 is able to move relative to the other surrounding apices 828.

In some embodiments, the chord 842 may be made of non-absorbable material such as nylon, silk, polypropylene, polyester, or others. In some embodiments, the chord 842 may be made of a bioresorbable material. By using a bioresorbable material, the chord 842 may serve its intended purpose during deployment at an implantation location. For example, the chord 842 may prevent asymmetrical expansion of the apices 828, excessive deflection of the apices 828, and/or excessively deep penetration of the apices 828. After the frame 800 is stabilized and in place, over time, the bioresorbable material may dissolve or otherwise cause the chord 842 to dissipate such that it is no longer attached to the frame 800. With the absence of the chord 842, any potential obstruction to blood flow caused by the chord 842 is removed as the chord is only temporarily part of the implant. Examples of such bioresorbable materials may include catgut (e.g., collagen derived from sheep intestinal submucosa), reconstituted collagen, polyglycolide (e.g., Dexon®, Dexon II®, Dexon S®), poly(glycolide/lactide) random copolymer (e.g., Vicryl®), antimicrobial-coated Vicryl® (Vicryl Plus®), poly-p-dioxanone (e.g., PDS®, PDSII® PDS Plus), poly(glycolide/trimethylene carbonate) block copolymer (e.g., Maxon®), poly(glycolide/ε-caprolactone) (e.g., Monocryl®, Monocryl Plus), poly(gycolide/p-dioxanone/trimethylene carbonate) triblock copolymer (e.g., Biosyn®), poly(glycolide/ε-caprolactone/trimethylene carbonate) triblock copolymer (Monosyn®), poly(glycolide/L-lactide/ε-caprolactone/trimethylene carbonate) teriblock copolymer (Caprosyn®), 100 % poly-L-lactide (Orthodek®), polydroxyalkanoates (e.g., poly-4-hydroxybuytrate (P4HB) such as TephaFlex®), and/or others.

In some embodiments, the chord 842 is made of an elastomeric material (which may or may not be bioresorbable) to facilitate adaptability of the frame 800 to the anatomy of the patient at the implantation location. For example, using an elastomeric material may permit the deformation, stretching, or other movement of the frame 800 by the apices 828 moving relative to each other more than is possible with a non-elastic material. By permitting some movement of the apices 828 relative to each other, the frame 800 may better accommodate variations in anatomy.

FIGS. 9A-9D illustrate various examples of an apex (such as apices 928a, 928b, and 934) associated with a chord (such as chord 942a, 942b, and 942c). FIG. 9A illustrates an example of the apex 928a with an eyelet 938a formed by strut-wrapping material 948a and/or 948b wrapped around terminal struts 925aa and 925ab. FIG. 9B illustrates an example of the apex 929b with an eyelet 938b formed as a hole in the apex 928b. FIG. 9C illustrates an example of the apex 934 of the connector tab 932 with the eyelet 933 formed by a gap 937 left by a sealing skirt 940. FIG. 9D illustrates an example of the apex 928a with an eyelet 938a formed by strut-wrapping material 948d.

With reference to FIG. 9A, the cell 924 may be similar or comparable to the cell 824, the terminal struts 925aa and 925ab may be similar or comparable to the terminal struts 825a and 825b, the apex 928a may be similar or comparable to the apices 828, the eyelet 938a may be similar or comparable to the eyelet 838, the chord 942a may be similar or comparable to the chord 842, and the strut-wrapping material 948a and 948b wrapped around the terminal struts 925aa and 925ab may be similar or comparable to the strut-wrapping material 848.

As illustrated in FIG. 9A, the strut-wrapping material 948a and 948b may facilitate the formation of the eyelet 938a. For example, as the strut-wrapping material 948a is wrapped along the terminal strut 925aa and as the strut-wrapping material 948b is wrapped along the terminal strut 925ab, at some point the strut-wrapping materials 948a and 948b begin to interfere with each other or otherwise overlap. However, this occurs before the terminal struts 925aa and 925ab meet to form the apex 928a. The space left between where the strut-wrapping materials 948a and 948b overlap and the two terminal struts 925aa and 925ab meet serves as the eyelet 938a.

In some embodiments, the strut-wrapping materials 948a and 948b may be spaced such that the strut-wrapping materials 948a and 948b are offset from each other while directly overlapping to form the eyelet 938a. Additionally or alternatively, the strut-wrapping materials 948a and 948b may be aligned with each other such that a first loop of the strut-wrapping material 948a corresponds with and is directly across from a second loop of the strut-wrapping material 948b. In such a configuration, the first loop and the second loop may compress against each other as the strut-wrapping materials 948a and 948b approach the eyelet 938a.

By forming the eyelet 938a without punching a hole or otherwise forming a hole in the apex 928a, the structural integrity of the apex 928a may be maintained.

With reference to FIG. 9B, the cell 924 may be similar or comparable to the cell 824, the terminal struts 925ba and 925bb may be similar or comparable to the terminal struts 825a and 825b, the apex 928b may be similar or comparable to the apices 828, the eyelet 938b may be similar or comparable to the eyelet 838, and the chord 942b may be similar or comparable to the chord 842.

As illustrated in FIG. 9B, the apex 928b may include a large enough footprint that the eyelet 938b may be punched or otherwise formed in the apex 928b. For example, when laser-cutting the frame, the eyelet 938b may be one of the portions cut or removed from an initial tube of material. Additionally or alternatively, the eyelet 938b maybe punched from the frame after the cells have been cut out.

In some embodiments, the footprint of the apex 928b may be large enough that the apex 928b includes as much material around the eyelet 938b as is present along the terminal struts 925ba and 925bb.

With reference to FIG. 9C, the cell 924 may be similar or comparable to the cell 824, the terminal struts 925ca and 925cb may be similar or comparable to the terminal struts 825a and 825b, the connector tab 932 may be similar or comparable to the connector tab 832, the eyelet 933 may be similar or comparable to the eyelet 833, the apex 934 maybe similar or comparable to the apex 834, the distance 936 maybe similar or comparable to the distance 836, the sealing skirt 940 may be similar or comparable to the sealing skirt 850, and the chord 942c may be similar or comparable to the chord 842.

As illustrated in FIG. 9C, the eyelet 933 may be formed by the gap 937 left between the sealing skirt 940 and the connector tab 932. For example, as the sealing skirt 940 is coupled to the terminal struts 925ca and 925cb (e.g., by being sewn onto the terminal struts 925ca and 925cb using strut-wrapping material 948c), the sealing skirt 940 may be positioned and cover a majority of the cell 924 while leaving the gap 937. In some embodiments, the eyelet 933 may be the distance 936 from the apex 934 of the connector tab 932.

In some embodiments, the strut-wrapping material 948c may attach the sealing skirt 940 to the terminal struts 925ca and 925cb at a same time or in a similar manufacturing step as when the chord 942c is threaded through the eyelet 933. For example, the chord 942c may be threaded through the cell 924 and the sealing skirt 940 may be positioned on the terminal struts 925ca and 925cb and sutured into place. In such an embodiment, the sealing skirt 940 may abut the chord 942c and/or be sutured into or against the chord 942c.

As illustrated in FIG. 9D, the strut-wrapping material 948d may facilitate the formation of the eyelet 938a. For example, the strut-wrapping material 948d may be wrapped along the terminal strut 925aa towards the apex 928a. When the wrapping of the strut-wrapping material 948d reaches the location of the eyelet 938a, the strut-wrapping material 948d may pass from the terminal strut 925aa and span over to the terminal strut 925ab and be wrapped along the terminal strut 925ab away from the apex 928a. In doing so, a single, continuous piece of strut-wrapping material 948 may be used to create the eyelet 938a by enclosing both the terminal strut 925aa and the terminal strut 925ab.

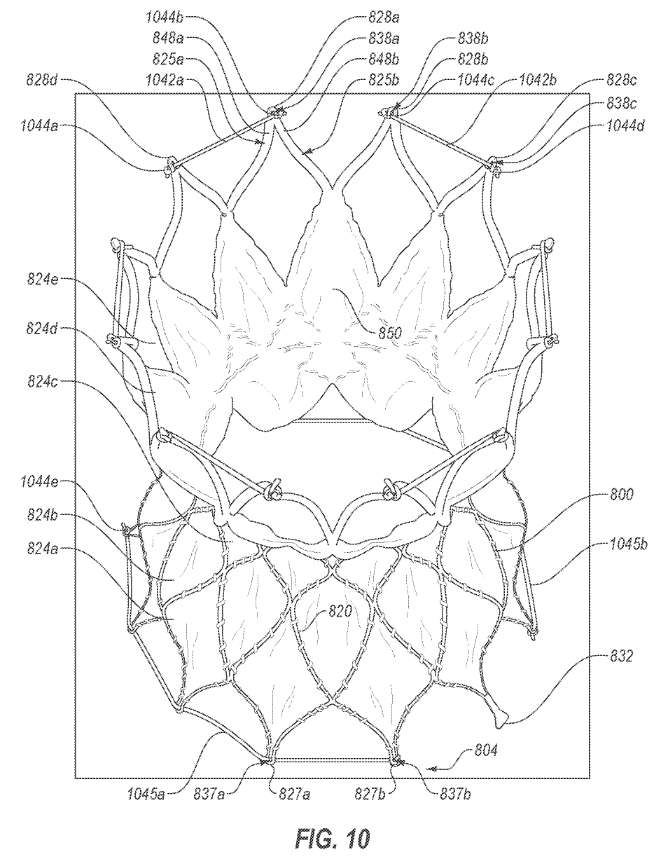

FIG. 10 is a perspective view of another example of the frame 800 with chords 1042 between apices 828. For convenience, the frame 800 may be the same or similar to that illustrated in FIG. 8 with similarly numbered components. As illustrated in FIG. 10, the chords 1042 may be discontinuous around the circumference of the frame 800.

The chords 1042 (such as the chords 1042a and/or 1042b) may connect two or more successive apices 828. For example, the chord 1042a may span between the apices 828d and 828a. In these and other embodiments, the chord 1042a may include a first knot 1044a and a second knot 1044b on the outsides of the eyelets 838. For example, the knot 1044b may be outside of the eyelet 838a on a side opposite from the apex 828d. By placing the knots 1044a and 1044b on the outside of the apices 828a, the chord 1042a may be fixed to the frame 800.

In some embodiments, the chord 1042a may be a continuation of a wrapping 848a about the terminal strut 825a. For example, the wrapping 848a may extend along the terminal strut 825a until the apex 828a is reached, after which the thread or suture of the wrapping 848a may progress from the eyelet 838a to the apex 828d and through the eyelet 838d. On the other side of the eyelet 838d, the knot 1044a may be tied to fix the chord 1042a to the frame 800.

In some embodiments, the chord 1042 may span a single pair of successive apices 828, such as is observed with the chords 1042a and 1042b. In these and other embodiments, by providing the chord 1042 in the disjointed manner, some or all of the benefits compared to the embodiment illustrated in FIG. 8 may be obtained. For example, even though disjointed, each of the apices 828 may have a chord 1042 coupled to at least one side of the apex, which may limit the amount of penetration permitted for the apices 828. Additionally or alternatively, the disjointed chords 1042 may permit greater flexibility such that the apices 828 are better able to conform to variations in the physiology of a patient. Additionally, while asymmetrical expansion of the frame 800 may be reduced, it might still be possible for sets of apices on opposite sides of the frame 800 to expand at different rates or in different amounts.

In some embodiments, the configuration of chords may be different at the first end 804 and the second end 808. For example, as illustrated in FIG. 10, the chords 1042 may be disjointed and coupled to every other apex 828 in a manner in which each apex 828 is coupled by the corresponding chord 1042 to one other apex 828 and/or in which every other span between apices 828 includes the corresponding chord 1042. The chords 1045 may be coupled to multiple apices 827 (such as the chord 1045a being coupled to five apices 827) and fixed to the frame 800 at either end of the multiple apices 827. In some embodiments, the chords 1045 may span the apices 827 between but not including the two connector tabs 832. For example, the first chord 1045a may extend between the apices 827 in one direction about the circumference of the frame from the connector tab 832, and the second chord 1045b may extend between the apices 827 in the other direction from the connector tab 832. In some embodiments, the configuration of chords may be the same at the first end 804 and the second end 808.

With reference to the first end 804, by including chords 1045 that span half or approximately half (e.g. one less than half, two less than half, etc.) of the apices 827 of the frame 800, the apices 827 may be better constrained to prevent asymmetrical expansion and/or to prevent or limit deflection when penetrating the tissue of the patient. In some embodiments, all of the apices 827 except the connector tabs 832 may be coupled to one or more of the chords 1045.

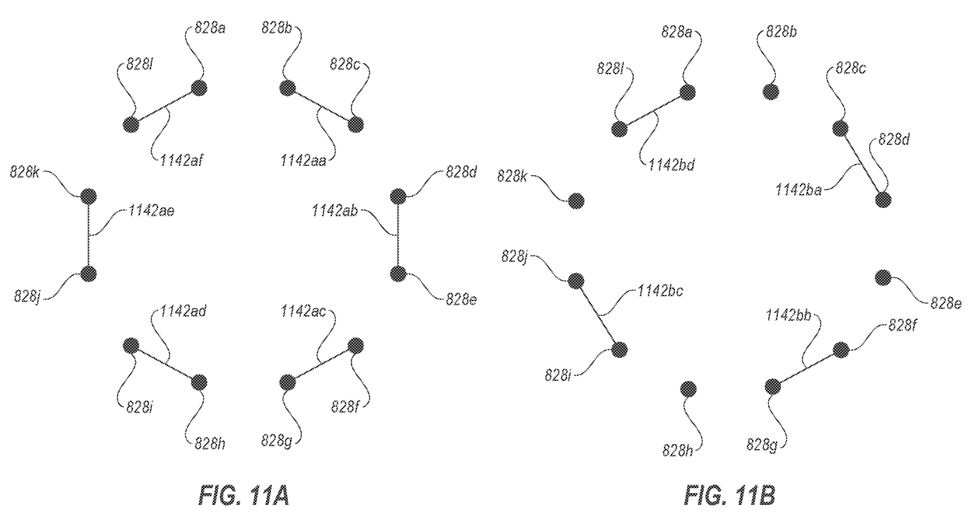

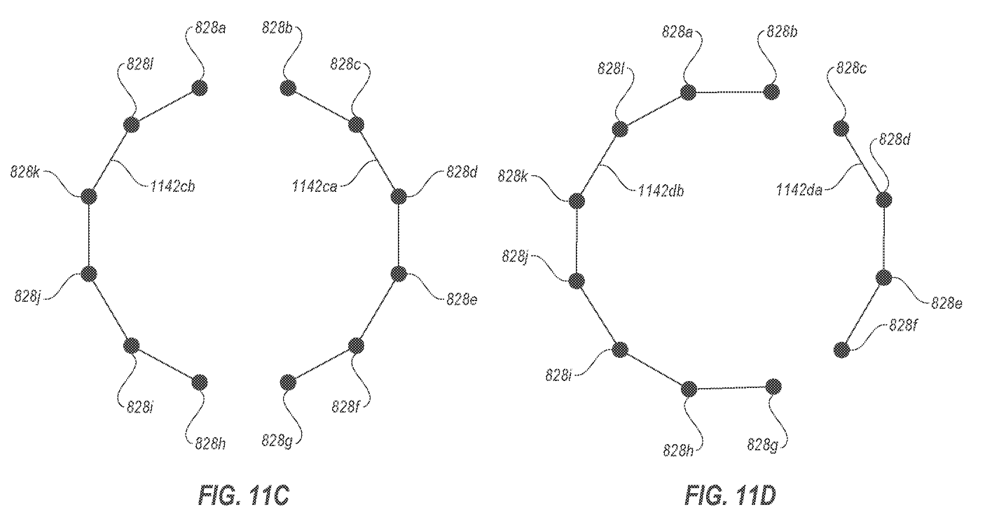

While two examples of configurations of chords are illustrated in FIGS. 8 and 10, it will be appreciated that any type or combination of configurations are contemplated in the present disclosure. Some other examples of configurations are illustrated in FIGS. 11A-11D, although it will be appreciated that other configurations are specifically contemplated. In these and other embodiments, any of the disclosed configurations may be used at the first end 804, the second end 804, or both. For example, the configuration illustrated at the second end 808 in FIG. 8 (e.g., all of the apices 828 connected by a single chord) and the configuration illustrated at the first end 804 in FIG. 10 (e.g., one less than half of the apices 827 on one side of the frame 800 are coupled to one chord and one less than half of the apices 827 on the other side are coupled to another chord) may be used in a single implementation. As another example, the configuration illustrated in FIG. 11A may be used at both the first end 804 and the second end 808.

FIGS. 11A-11D illustrate various example configurations of chords 1142 and apices 828 of a docking station. FIG. 11A illustrates a configuration in which every other span between apices 828 includes a chord 1142. FIG. 11B illustrates a configuration in which every third span between apices 828 includes a chord 1142. FIG. 11C illustrates an embodiment in which two chords 1142 are used to connect the apices, with half of the apices 828 being connected to each of the chords 1142. FIG. 11D illustrates an embodiment in which two chords 1142 are used to couple the apices 828, although the two chords 1142 are different lengths. While illustrated as using the apices 828, it will be appreciated that the nodes of the configurations are equally applicable to the apices 827 and/or the connector tab 832.

As illustrated in FIG. 11A, the chord 1142aa spans between apices 828b and 828c, the chord 1142ab spans between apices 828d and 828e, the chord 1142ac spans between apices 828f and 828g, the chord 1142ad spans between apices 828h and 828i, the chord 1142ae spans between apices 828j and 828k, and the chord 1142af spans between apices 828l and 828a. As illustrated in FIG. 11A, in some embodiments, every other span of apices 828 include a chord 1142 spanning therebetween. By doing so, each apex 828 includes one chord attached thereto. This reduces penetration of the apices 828 while still allowing some flexibility of the implant to move and adapt to variations in anatomy of the patient. For example, the apices 828b and 828c may be coupled via the chord 1142aa such that the two apices 828b and 828c have their expansion tied together. For example, the apex 828b cannot expand excessively beyond the expansion of the apex 828c. While the apices 828b and 828c together may expand more broadly than the other apices, their expansion is still constrained to a certain degree. Additionally, each of the apices 828 may include at least a certain level of support to reduce deflection of the apices 828. For example, the apex 828b, via the chord 1142aa, may mitigate deflection of the apex 828c.

As illustrated in FIG. 11B, the chord 1142ba spans between apices 828c and 828d, the chord 1142bb spans between apices 828f and 828g, the chord 1142bc spans between apices 828j and 828i, and the chord 1142bd spans between apices 828l and 828a. As illustrated in FIG. 11B, in some embodiments, every third span of apices 828 include a chord 1142 spanning therebetween. By doing so, the apices 828 include greater freedom to expand and deform to accommodate variations in anatomy. Additionally, there is a certain amount of resistance to penetration of the apices 828, although some apices (such as apices 828b, 828e, 828h, and 828k) are free-floating and thus able to penetrate more deeply. There is a certain amount of reduction in penetration due to the adjacent apices of the free apices (such as apices 828a and 828c adjacent to the free apex 828b) being constrained by chords 1142.

As illustrated in FIG. 11C, the chord 1142ca spans between apices 828b, 828c, 828d, 828e, 828f, and 828g, and the chord 1142cb spans between apices 828h, 828i, 828j, 828k, 828l, and 828a. As illustrated in FIG. 11C, each of the chords 1142ca and 1142cb are coupled to half of the apices, while leaving a gap between the two chords 1142ca and 1142cb (e.g., the gap between the apices 828a and 828b and the gap between the apices 828h and 828g). By doing so, the apices 828 are generally constrained but with some flexibility between how the two halves of the implant may expand.

As illustrated in FIG. 11D, the chord 1142da spans between apices 828c, 828d, 828e, and 828f, and the chord 1142db spans between apices 828g, 828h, 828i, 828j, 828k, 828l, 828a, and 828b. As illustrated in FIG. 11D, the chords 1142da and 1142db are asymmetrically coupled to the apices. By doing so, the expansion of the apices 828 may occur in a slightly asymmetrical manner. For example, apices 828c, 828d, 828e, and 828f may be able to expand more broadly and/or with greater flexibility due to the shorter chord 1142da while the apices 828g, 828h, 828i, 828j, 828k, 828l, 828a, and 828b may be more constrained via the longer chord 1142db. In some embodiments, the potential for asymmetrical expansion may be oriented in a certain direction to accommodate various aspects of the anatomy of the patient. In these and other embodiments, the frame may include one or more radiopaque markers to facilitate orientation and/or guidance of the frame during a procedure. For example, the radiopaque markers may include tantalum, bismuth, iodine, barium, or gold coined into one or more of the apices, such as the apices at the ends of the chords.

FIGS. 12A and 12B illustrate another example implementation of a frame 1200 (or stent) that includes strut-wrapping material 1248 enclosing terminal struts 1225 of the frame 1200. FIG. 12B is a closeup of one of the apices 1228 of FIG. 12A. The frame 1200 may form a body of a docking station that may be similar or comparable to the frame 100 and/or 800. The frame 1200 may include a first end 1204 and a second end 1208, which may be similar or comparable to the first ends 104 and 804 and the second ends 108 and 808. In some examples, the first end 1204 may be an inflow end, and the second end 1208 may be an outflow end. In some examples, the first end 1204 may be an outflow end, and the second end 1208 may be an inflow end. The terms “inflow” and “outflow” are related to the normal direction of blood flow (e.g., antegrade blood flow) through the frame. In the unconstrained, expanded state of the frame 1200 shown in FIG. 12A, the frame 1200 forms a valve seat within which a valve may be deployed into the frame 1200.

The frame 1200 may include struts 1220 (which may be similar or comparable to the struts 120 and 820) which form cells 1224 (which may be similar or comparable to the cells 124 and/or 824), such as the rows of cells 1224a-1224e. At the first end 1204 of the frame 1200, the struts 1220 may form apices 1227, such as the apices 1227a and 1227b. At the second end 1208 of the frame 1200, the struts 1220 may form apices 1228, such as the apices 1228a and 1228b. The apices 1227 and 1228 maybe similar or comparable to the apices 128, 827, and/or 828. With reference to the second end 1208, terminal struts 1225 may come together to form the apices 1228 (such as the terminal struts 1225a and 1225b coming together to form the apex 1228a and the terminal struts 1225c and 1225d coming together to form the apex 1228b). In some embodiments the apices 1227 and 1228 may be characterized as inflow apices or outflow apices, depending on the orientation of the frame 1200 relative to the direction of blood flow.

The frame 1200 may include one or more shoulders 1222 where the struts 1220 meet in the frame 1200, such as an intersection between cells 1224. For example, the shoulders 1222a, 1222b, 1222c, and 1222d may represent intersections in the frame 1200 where the struts 1220 meet each other. Giving a specific example, the shoulder 1222d is the point at which the terminal struts 1225b and 1225c meet in a lateral corner of a cell in the row of cells 1224e.

The frame 1200 may include a sealing skirt 1240 disposed within the frame 1200. The sealing skirt 1240 maybe similar or comparable to the sealing skirt 140 and/or 850. In some embodiments, the sealing skirt 1240 may span some or all of the cells 1224. For example, the sealing skirt 1240 may fill the rows of cells 1224a, 1224b, 1224c, and 1224d but may not extend into the row of cells 1224e (e.g., the distal-most row of cells). In such an embodiment, the terminal struts 1225 may not have any material of the sealing skirt 1240 and/or sutures 1241 for fixing the sealing skirt 1240 to the frame 1200 associated therewith.

As illustrated in FIGS. 12A and 12B, the strut-wrapping material 1248 may create an enclosing layer around the terminal struts 1225 while leaving the apex 1228 exposed. By providing the strut-wrapping material 1248, an amount of penetration of the apices 1228 into the tissue of the patient at the implantation location may be controlled. For example, at the transition between an exposed portion 1264 of the terminal strut 1225a and an enclosed portion 1262 of the terminal strut 1225a, the strut-wrapping material 1248a may create a lip 1246 that may act to resist penetration of the apex 1228a into the tissue. By controlling a depth of penetration of the apices 1228, an amount of damage and/or tissue erosion may be reduced.

In some embodiments, the strut-wrapping material 1248 may extend from the exposed portion 1264 along the enclosed portion 1262 of the terminal strut 1225b up to the shoulder 1222d. Additionally or alternatively, the strut-wrapping material 1248 may extend beyond the shoulder 1222d such that the struts 1220 are enclosed even in regions without the sealing skirt 1240. By doing so, tissue erosion and other problems which may be caused by exposure of the tissue to metal or other more abrasive materials may be alleviated by the tissue being in contact with the strut-wrapping material 1248 rather than the struts 1220 of the frame 1200.

In some embodiments, the strut-wrapping material 1248 may be used to enclose any exposed portion of the struts 1220. For example, in some embodiments, the sealing skirt 1240 may fill fewer cells 1224 than illustrated in FIG. 12A, and a portion of or all of the exposed struts 1220 may be enclosed by the strut-wrapping material 1248.

In some embodiments, such as that illustrated in FIG. 12A, the apices 1228 about an entire circumference of the frame 1200 may include the strut-wrapping material 1248. While each individual apex 1228 with strut-wrapping material 1248 enclosing the terminal struts 1225 leading up thereto may not completely reduce the tissue penetration, the aggregate effect of all or most of the apices 1228 including the strut-wrapping material 1248 may collectively provide the desired amount of resistance to penetration.

In some embodiments, the strut-wrapping material 1248 may include a material used in sutures. For example, the strut-wrapping material 1248 may include an extension of the sutures 1241 used to fix the sealing skirt 1240 to the frame 1200. For example, as the sutures 1241 fix the sealing skirt 1240 to the frame in the row of cells 1224d approaching the shoulder 1222d, the sutures 1241 may then be wrapped about the terminal strut 1225b and/or 1225c. Additionally or alternatively, the strut-wrapping material 1248 may be made of a same substance as the sutures 1241 while being a separate strand or thread.

In some embodiments, the strut-wrapping material 1248 may include a fabric. For example, the strut-wrapping material 1248 may include an extension of the fabric used for the sealing skirt 1240. For example, a narrow strip or other portion of the sealing skirt 1240 may be wrapped about the terminal strut 1225. Additionally or alternatively, the strut-wrapping material 1248 may be made of a same fabric as the sealing skirt 1240 while being a separate strand or piece of fabric from the sealing skirt 1240.

In some embodiments, the strut-wrapping material 1248 may include a biomaterial, such as human tissue or artificial tissue, to facilitate biocompatibility of the exposed struts 1220. Such human tissue may be tissue harvested elsewhere from the patient, from a cadaver, tissue engineered or grown, or any other source of tissue. In some embodiments, the strut-wrapping material 1248 may include a scaffolding or other structure embedded with the tissue.

In some embodiments, the strut-wrapping material 1248 may be made of the same substance as the chords 842, 845, 942, 1042, 1045, and/or 1142. In some embodiments, the strut-wrapping material 1248 may be used in conjunction with the chords 842, 845, 942, 1042, 1045, and/or 1142. For example, FIG. 8 illustrates an example in which the strut-wrapping material 848 is used in conjunction with the chord 842, which may facilitate resistance of penetration and/or greater biocompatibility.

While illustrated as being disposed on the apices 1228 at the second end 1208, it will be appreciated that the strut-wrapping material 1248 may also be disposed on the struts 1220 leading to the apices 1227 at the first end 1204. In such an embodiment, the strut-wrapping material 1248 at the first end 1204 may be made of a first substance and the strut-wrapping material 1248 at the second end 1208 may be made of a second substance that is the same as or different from the first substance. For example, the strut-wrapping material 1248 at the first end 1204 may be made of human tissue and the strut-wrapping material 1248 at the second end 1208 may be made of sutures wrapped about the terminal struts 1225.

Any of the various systems, devices, apparatuses, etc. in this disclosure may be sterilized (e.g., with heat, radiation, ethylene oxide, hydrogen peroxide, etc.) to ensure they are safe for use with patients, and the methods herein may comprise sterilization of the associated system, device, apparatus, etc. (e.g., with heat, radiation, ethylene oxide, hydrogen peroxide, etc.).

The treatment techniques, methods, steps, etc. described or suggested herein or in references incorporated herein may be performed on a living animal or on a non-living simulation, such as on a cadaver, cadaver heart, anthropomorphic ghost, simulator (e.g., with the body parts, tissue, etc. being simulated), etc.

### Additional Examples of the Disclosed Technology

In view of the above-described implementations of the disclosed subject matter, this application discloses the additional examples enumerated below. It should be noted that one feature of an example in isolation or more than one feature of the example taken in combination and, optionally, in combination with one or more features of one or more further examples are further examples also falling within the disclosure of this application.

Example 1. A docking station for a prosthetic implant, comprising:
a frame comprising a plurality of struts, wherein the plurality of struts form a set of apices; and
a chord of material spanning between at least two apices of the set of apices.

Example 2. Any of the foregoing examples, such as example 1, wherein the chord of material is coupled to the at least two apices at respective eyelets of the at least two apices.

Example 3. Any of the foregoing examples, such as example 2, wherein the respective eyelets are formed by strut-wrapping material wrapped around the plurality of struts.

Example 4. Any of the foregoing examples, such as example 3, wherein the strut-wrapping material is wrapped around individual terminal struts of the plurality of struts.

Example 5. Any of the foregoing examples, such as example 2, wherein the respective eyelets are formed as holes in the frame at the at least two apices.

Example 6. Any of the foregoing examples, such as any of examples 1-5, wherein the chord is a first chord and the at least two apices are a first set of apices, the docking station further comprising a second chord distinct from the first chord and spanning between a second set of apices.

Example 7. Any of the foregoing examples, such as example 6, wherein the second set of apices include less than half of all of the apices of the docking station.

Example 8. Any of the foregoing examples, such as example 6, wherein the second set of apices include half of all of the apices of the docking station.

Example 9. Any of the foregoing examples, such as example 6, wherein the second set of apices include more than half of all of the apices of the docking station.

Example 10. Any of the foregoing examples, such as any of examples 6-9, further comprising a third chord distinct from the first chord and the second chord and spanning between a third set of apices.

Example 11. Any of the foregoing examples, such as example 10, wherein the first chord, the second chord, and the third chord are all disposed at an inflow end of the frame.

Example 12. Any of the foregoing examples, such as example 10, wherein the first chord, the second chord, and the third chord are all disposed at an outflow end of the frame.

Example 13. Any of the foregoing examples, such as examples 6, 7, or 10-12, wherein every other gap between successive apices includes a respective chord.

Example 14. Any of the foregoing examples, such as examples 6, 7, or 10-12, wherein every third gap between successive apices includes a respective chord.

Example 15. Any of the foregoing examples, such as any of examples 1-12 or 14, wherein the set of apices includes at least one apex that does not have a chord of material coupled thereto.

Example 16. Any of the foregoing examples, such as any of examples 1-15, further comprising radiopaque markers.

Example 17. Any of the foregoing examples, such as any of examples 1-16, wherein the set of apices includes inflow apices at an inflow end of the frame and outflow apices at an outflow end of the frame.

Example 18. Any of the foregoing examples, such as example 17, wherein the at least two apices are included in the inflow apices and a second set of at least two apices of the outflow apices include a second chord of material spanning between the second set of at least two apices.

Example 19. Any of the foregoing examples, such as any of examples 17-18, wherein all of the inflow apices are successively coupled together via the chord.

Example 20. Any of the foregoing examples, such as any of examples 17-19, wherein all of the outflow apices are successively coupled together via a second chord of material.

Example 21. Any of the foregoing examples, such as any of examples 17-20, wherein a first configuration of chords associated with the inflow apices, including the chord of material spanning between the at least two apices, is a same configuration as a second configuration of chords associated with the outflow apices.

Example 22. Any of the foregoing examples, such as any of examples 17-20, wherein a first configuration of chords associated with the inflow apices, including the chord of material spanning between the at least two apices, is a different configuration than a second configuration of chords associated with the outflow apices.

Example 23. Any of the foregoing examples, such as any of examples 1-22, wherein a first apex of the set of apices includes a connector tab.

Example 24. Any of the foregoing examples, such as example 23, wherein the at least two apices includes the first apex such that the chord of material is coupled to the first apex.

Example 25. Any of the foregoing examples, such as example 23, wherein the chord of material is not coupled to the first apex.

Example 26. Any of the foregoing examples, such as any of examples 1-25, further comprising a sealing skirt filling one or more rows of cells formed by the frame.

Example 27. Any of the foregoing examples, such as example 26, wherein the sealing skirt fills up to but excludes a distal-most row of cells formed by the frame.

Example 28. Any of the foregoing examples, such as example 27, wherein the at least two apices extend from the distal-most row of cells.

Example 29. Any of the foregoing examples, such as example 26, wherein the sealing skirt fills a distal-most row of cells formed by the frame.

Example 30. Any of the foregoing examples, such as example 29, wherein the at least two apices extend from the distal-most row of cells.

Example 31. Any of the foregoing examples, such as any of examples 1-30, wherein the chord of material includes an elastic material.

Example 32. Any of the foregoing examples, such as any of examples 1-31, wherein the chord of material includes a material used in sutures.

Example 33. Any of the foregoing examples, such as any of examples 1-32, wherein the chord of material includes a bioresorbable material.

Example 34. A method comprising:
obtaining a frame, the frame comprising a plurality of struts, wherein the plurality of struts form a set of apices;
threading a chord between eyelets of at least two apices of the set of apices; and
coupling the chord to the frame.

Example 35. Any of the foregoing examples, such as example 34, further comprising wrapping portions of individual struts of the plurality of struts with strut-wrapping material to form the eyelets of the at least two apices.

Example 36. Any of the foregoing examples, such as any of examples 34-35, further comprising punching holes in the at least two apices to form the eyelets.

Example 37. Any of the foregoing examples, such as any of examples 34-36, wherein coupling the chord to the frame includes tying the chord to one of the at least two apices.

Example 38. Any of the foregoing examples, such as any of examples 34-36, wherein coupling the chord to the frame includes tying a first end of the chord to a second opposite end of the chord after threading the chord through the eyelets of the at least two apices.

Example 39. Any of the foregoing examples, such as any of examples 34-38, wherein the set of apices includes inflow apices at an inflow end of the frame and outflow apices at an outflow end of the frame, the at least two apices included in the inflow apices and a second set of at least two apices included in the outflow apices; and wherein the method further comprises:
threading a second chord of material through the second set of at least two apices; and coupling the second chord of material to the frame.

Example 40. Any of the foregoing examples, such as examples 34-39, wherein the chord includes a first chord of material, the method further comprising:
threading a second chord of material distinct from the first chord through a second set of apices distinct from the at least two apices; and
coupling the second chord of material to the frame.

Example 41. Any of the foregoing examples, such as example 40, further comprising:
threading a third chord of material distinct from the first chord and the second through a third set of apices; and
coupling the third chord to the frame.

Example 42. Any of the foregoing examples, such as example 41, wherein the first chord, the second chord, and the third chord are all disposed at an inflow end of the frame.

Example 43. Any of the foregoing examples, such as example 41, wherein the first chord, the second chord, and the third chord are all disposed at an outflow end of the frame.

Example 44. A docking station for a prosthetic implant, comprising:
a frame comprising a plurality of struts, the plurality of struts forming apices; and
strut-wrapping material enclosing two terminal struts of the plurality of struts, the two terminal struts leading up to an apex of the apices formed by the two terminal struts, the strut-wrapping material leaving the apex exposed.

Example 45. Any of the foregoing examples, such as example 44, wherein the strut-wrapping material forms a lip at a transition from an enclosed portion of a given strut of the two terminal struts to an exposed portion of the given strut.

Example 46. Any of the foregoing examples, such as any of examples 44-45, wherein the strut-wrapping material includes a substance used in sutures wrapped individually about the two terminal struts.

Example 47. Any of the foregoing examples, such as any of examples 44-45, wherein the strut-wrapping material includes a fabric wrapped individually about the two terminal struts.

Example 48. Any of the foregoing examples, such as any of examples 44-45, wherein the strut-wrapping material includes a biomaterial.

Example 49. Any of the foregoing examples, such as example 48, wherein the biomaterial includes human tissue.

Example 50. Any of the foregoing examples, such as any of examples 44-45, wherein the strut-wrapping material includes artificial tissue.

Example 51. Any of the foregoing examples, such as any of examples 44-50, wherein the strut-wrapping material covers at least one shoulder at an intersection of two struts of the plurality of struts.

Example 52. Any of the foregoing examples, such as example 51, wherein the shoulder is located in a lateral corner of a cell formed by the frame.

Example 53. Any of the foregoing examples, such as example 52, wherein the cell is formed at least in part by the terminal struts.

Example 54. Any of the foregoing examples, such as example 51-53, wherein the strut-wrapping material extends from an exposed portion of a given strut of the two terminal struts to at least the shoulder.

Example 55. Any of the foregoing examples, such as example 54, wherein the strut-wrapping material extends beyond the shoulder in a direction away from the apex.

Example 56. Any of the foregoing examples, such as any of examples 44-55, wherein the apices include inflow apices at an inflow end of the frame and outflow apices at an outflow end of the frame.

Example 57. Any of the foregoing examples, such as example 54, wherein the apex is included in the inflow apices and a second strut-wrapping material encloses two outflow terminal struts leading to an outflow apex at the outflow end of the frame while leaving the outflow apex exposed.

Example 58. Any of the foregoing examples, such as example 57, wherein the second strut-wrapping material and the strut-wrapping material are made of a same type of substance.

Example 59. Any of the foregoing examples, such as example 57, wherein the second strut-wrapping material and the strut-wrapping material are made of a different type of substance.

Example 60. Any of the foregoing examples, such as any of examples 44-57, wherein for each of the apices about an entire circumference of the frame, respective strut-wrapping material encloses respective terminal struts leading up to respective apices while leaving the respective apices exposed; and wherein the respective strut-wrapping material includes the strut-wrapping material, the respective terminal struts include the two terminal struts, and the respective apices include the apex.

Example 61. Any of the foregoing examples, such as any of examples 44-57, further comprising a chord of material spanning between the apex and a second adjacent apex of the apices.

Example 62. Any of the foregoing examples, such as example 61, wherein the chord of material spans between all of the apices about an entire circumference of the frame.

Example 63. Any of the foregoing examples, such as any of examples 61-62, wherein the chord of material is threaded through an eyelet in the exposed portion of the apex.

Example 64. Any of the foregoing examples, such as example 63, wherein the eyelet is formed as a gap left without the strut-wrapping material at exposed portions of the two terminal struts.

Example 65. Any of the foregoing examples, such as example 63, wherein the eyelet is formed as a hole in the apex.

Example 66. Any of the foregoing examples, such as any of examples 3-4, wherein the strut-wrapping material is a continuous piece of material wrapped around at least two successive terminal struts of the plurality of struts.

Example 67. Any of the foregoing examples, such as example 36, wherein wrapping the portions of the individual struts includes wrapping a continuous piece of material along a first terminal strut, across to a successive second terminal strut, and along the second terminal strut to form a given eyelet of the eyelets.

Example 68. Any of the foregoing examples, such as any of examples 44-65, wherein the strut-wrapping material includes a continuous piece of strut-wrapping material enclosing and spanning between the two terminal struts leading up to the apex.

Example 69. A method comprising:
obtaining a frame, the frame comprising a plurality of struts, wherein the plurality of struts form a set of apices; and
wrapping portions of two terminal struts of the plurality of struts with strut-wrapping material, the two terminal struts leading up to an apex of the set of apices formed by the two terminal struts, the strut-wrapping material leaving the apex exposed.

Example 70. Any of the foregoing examples, such as example 69, wherein wrapping the portions of the two terminal struts includes wrapping a continuous piece of material along a first terminal strut of the two terminal struts, across to a successive second terminal strut of the two terminal struts, and along the second terminal strut to form an eyelet.

Example 71. Any of the foregoing examples, such as example 69 or 70, wherein the strut-wrapping material forms a lip at a transition from an enclosed portion of a given strut of the two terminal struts to an exposed portion of the given strut.

Example 72. Any of the foregoing examples, such as any one of examples 69-71, wherein wrapping the portions of the two terminal struts with the strut-wrapping material includes wrapping the portions of the two terminal struts with at least one of:
a substance used in sutures;
a fabric;
a biomaterial;
human tissue; or
an artificial tissue substance.

Example 73. Any of the foregoing examples, such as any one of examples 69-72, further comprising, wrapping portions of all terminal struts of the plurality of struts with the strut-wrapping material.

Example 74. Any of the foregoing examples, such as any one of examples 69-73, wherein wrapping the portions of the two terminal struts with the strut-wrapping material includes wrapping, with the strut-wrapping material, at least one shoulder at an intersection of one of the two terminal struts with another terminal strut of the plurality of struts.

Example 75. Any of the foregoing examples, such as example 74, wherein wrapping the portions of the two terminal struts with the strut-wrapping material includes wrapping the one of the two terminal struts with the strut-wrapping material from the apex to and including the shoulder.

Example 76. Any of the foregoing examples, such as example 75, wherein the strut-wrapping material extends beyond the shoulder in a direction away from the apex.

Example 77. Any of the foregoing examples, such as any one of examples 69-76, wherein:
the apices include inflow apices at an inflow end of the frame and outflow apices at an outflow end of the frame;
the apex led up to by the two terminal struts is an inflow apex of the inflow apices;
the method further comprises wrapping portions of two other terminal struts of the plurality of struts with second strut-wrapping material, the two other terminal struts leading up to an outflow apex of the outflow apices, the second strut-wrapping material leaving the outflow apex exposed.

Example 78. Any of the foregoing examples, such as any one of examples 69-77, further comprising:
threading a chord between eyelets of at least two apices of the set of apices; and
coupling the chord to the frame.

Example 79. Any of the foregoing examples, such as example 78, further comprising threading the chord between all other eyelets of all other apices of the set of apices at an inflow end or outflow end of the frame such that the chord extends around an entire circumference of the frame.

Example 80. Any of the foregoing examples, such as example 78 or 79, further comprising, forming an eyelet in the apex by wrapping the strut-wrapping material around portions of each of the two terminal struts up to a point at which the strut-wrapping material of one of the two terminal struts touches or overlaps the strut-wrapping material of the other of the two terminal struts to form a gap in the exposed portion of the apex, the eyelet including the gap.

Example 81. Any of the foregoing examples, such as example 78 or 79, further comprising forming an eyelet in the apex by wrapping the strut-wrapping material as a continuous piece of material around portions of the two terminal struts and spanning a gap between the two terminal struts near the apex from one terminal strut to the other.

Example 82. Any of the foregoing examples, such as example 78 or 79, further comprising forming an eyelet in the apex by forming a hole in the apex.

The features described herein with regard to any example may be combined with other features described in any one or more of the other examples, unless otherwise stated.

In view of the many possible ways in which the principles of the disclosure may be applied, it should be recognized that the illustrated configurations depict examples of the disclosed technology and should not be taken as limiting the scope of the disclosure nor the claims. Rather, the scope of the claimed subject matter is defined by the following claims and their equivalents.

What is claimed is:

1. A docking station for a prosthetic implant, comprising:
a frame comprising a plurality of struts, wherein the plurality of struts form a set of apices; and
a chord of material spanning between at least two apices of the set of apices.

2. The docking station of claim 1, wherein the chord of material is coupled to the at least two apices at respective eyelets of the at least two apices.

3. The docking station of claim 2, wherein the respective eyelets are formed by strut-wrapping material wrapped around the plurality of struts.

4. The docking station of claim 3, wherein the strut-wrapping material is wrapped around individual terminal struts of the plurality of struts.

5. The docking station of claim 3, wherein the strut-wrapping material is a continuous piece of material wrapped around at least two successive terminal struts of the plurality of struts.

6. The docking station of claim 1, wherein the chord is a first chord and the at least two apices are a first set of apices, the docking station further comprising a second chord distinct from the first chord and spanning between a second set of apices.

7. The docking station of claim 6, wherein the second set of apices include less than half of all of the apices of the docking station.

8. The docking station of claim 6, further comprising a third chord distinct from the first chord and the second chord and spanning between a third set of apices.

9. The docking station of claim 7, wherein every other gap between successive apices includes a respective chord.

10. The docking station of claim 7, wherein every third gap between successive apices includes a respective chord.

11. A docking station for a prosthetic implant, comprising:
a frame comprising a plurality of struts, the plurality of struts forming apices; and
strut-wrapping material enclosing two terminal struts of the plurality of struts, the two terminal struts leading up to an apex of the apices formed by the two terminal struts, the strut-wrapping material leaving the apex exposed.

12. The docking station of claim 11, wherein the strut-wrapping material forms a lip at a transition from an enclosed portion of a given strut of the two terminal struts to an exposed portion of the given strut.

13. The docking station of claim 11, wherein the strut-wrapping material covers at least one shoulder at an intersection of two struts of the plurality of struts.

14. The docking station of claim 13, wherein the shoulder is located in a lateral corner of a cell formed by the frame.

15. The docking station of claim 14, wherein the cell is formed at least in part by the terminal struts.

16. The docking station of claim 15, wherein the strut-wrapping material extends from an exposed portion of a given strut of the two terminal struts to at least the shoulder.

17. The docking station of claim 16, wherein the strut-wrapping material extends beyond the shoulder in a direction away from the apex.

18. The docking station of claim 11, wherein the apices include inflow apices at an inflow end of the frame and outflow apices at an outflow end of the frame.

19. The docking station of claim 18, wherein the apex is included in the inflow apices and a second strut-wrapping material encloses two outflow terminal struts leading to an outflow apex at the outflow end of the frame while leaving the outflow apex exposed.

20. The docking station of claim 19, wherein the second strut-wrapping material and the strut-wrapping material are made of a same type of substance.
